(19) United States
(12) Patent Application Publication
(10) Pub. No.: US 2026/0090742 A1
(43) Pub. Date: Apr. 2, 2026

(54) PULSE OXIMETER

(71) Applicant: SEIKO EPSON CORPORATION, Tokyo (JP)

(72) Inventor: Akira IKEDA, CHINO-SHI (JP)

(73) Assignee: SEIKO EPSON CORPORATION, Tokyo (JP)

(21) Appl. No.: 19/337,941

(22) Filed: Sep. 24, 2025

(30) Foreign Application Priority Data
Sep. 27, 2024 (JP) 2024-168888

(51) Int. Cl. A61B 5/1455; A61B 5/00

(52) U.S. Cl. CPC A61B 5/14552; A61B 5/7235; A61B 5/7271; A61B 5/742; A61B 5/746; A61B 2562/0238

(57) ABSTRACT

A pulse oximeter includes a saturation measurement unit that measures a blood oxygen saturation of a subject by a red light sensor and an infrared light sensor, a detection indicator calculation unit that calculates a detection indicator, and an abnormal value detection unit that detects an abnormal value of the oxygen saturation based on the detection indicator. The detection indicator calculation unit calculates, as the detection indicator, at least one of a green-red signal ratio that is a ratio between a green light pulse wave signal intensity ratio and a red light pulse wave signal intensity ratio, and a green-infrared signal ratio that is a ratio between the green light pulse wave signal intensity ratio and an infrared light pulse wave signal intensity ratio.

The present application is based on, and claims priority from JP Application Serial Number 2024-168888, filed September 27, 2024, the disclosure of which is hereby incorporated by reference herein in its entirety.

### BACKGROUND

### 1. Technical Field

The present disclosure relates to a pulse oximeter.

### 2. Related Art

A pulse oximeter measures an arterial blood oxygen saturation (SpO2) by irradiating a finger with red light and near infrared light sources and detecting light transmitted through the finger (see JP-A-2004-337605). When an oxygen desaturation index, which is an indicator of sleep apnea syndrome, is tested, the arterial blood oxygen saturation of a subject during sleep is measured by the pulse oximeter.

JP-A-2004-337605 is an example of the related art.

When measuring the arterial blood oxygen saturation of the subject during sleep, there is a problem that the oxygen saturation measured based on a photoplethysmogram exhibits an abnormal value that is low even though a detection state of the photoplethysmogram of detection light is normal in the pulse oximeter when the subject adopts a specific posture.

The specific posture includes, for example, a posture in which the subject lies supine on an elevated bed and a tip of a hand hangs downward below a height of the supine position. When the oxygen saturation is measured with the pulse oximeter worn on a wrist in such a posture, a low abnormal value may be exhibited relative to a measured value in a normal posture in which an arm does not hang down.

Since the abnormal value with the decreased oxygen saturation in such a specific posture occurs with pulsation as in a normal photoplethysmogram, the abnormal value cannot be distinguished from an arterial wave reflecting a normal oxygen saturation, and there is a problem that the abnormal value is confused with a decrease in the oxygen saturation due to sleep apnea.

### SUMMARY

A pulse oximeter according to a first aspect of the disclosure includes: a green light sensor configured to irradiate a subject with green light and detect the green light transmitted through or reflected from the subject; a red light sensor configured to irradiate the subject with red light and detect the red light transmitted through or reflected from the subject; an infrared light sensor configured to irradiate the subject with infrared light and detect the infrared light transmitted through or reflected from the subject; and a control device configured to process detection signals from the green light sensor, the red light sensor, and the infrared light sensor, in which the control device includes a saturation measurement unit that generates a measurement data group indicating an oxygen saturation of the subject based on the detection signals from the red light sensor and the infrared light sensor, a detection indicator calculation unit that calculates, as a detection indicator, a ratio between the detection signals from the green light sensor, the red light sensor, and the infrared light sensor, and an abnormal value detection unit that detects an abnormal value of the oxygen saturation based on the detection indicator, and the detection indicator calculation unit calculates, as the detection indicator, at least one of a green-red signal ratio that is a ratio between a green light pulse wave signal intensity ratio, which indicates a ratio of an amplitude of a vibration component in the detection signal output by the green light sensor to a signal intensity of the detection signal, and a red light pulse wave signal intensity ratio, which indicates a ratio of an amplitude of a vibration component in the detection signal output by the red light sensor to a signal intensity of the detection signal, and a green-infrared signal ratio that is a ratio between the green light pulse wave signal intensity ratio and an infrared light pulse wave signal intensity ratio, which indicates a ratio of an amplitude of a vibration component in the detection signal output by the infrared light sensor to a signal intensity of the detection signal.

A pulse oximeter according to a second aspect of the disclosure includes: a green light sensor configured to irradiate a subject with green light and detect the green light transmitted through or reflected from the subject; a red light sensor configured to irradiate the subject with red light and detect the red light transmitted through or reflected from the subject; an infrared light sensor configured to irradiate the subject with infrared light and detect the infrared light transmitted through or reflected from the subject; a control device configured to process detection signals from the green light sensor, the red light sensor and the infrared light sensor; and a display unit configured to display information obtained by the control device, in which the control device is configured to generate a measurement data group indicating an oxygen saturation of the subject based on the detection signals from the red light sensor and the infrared light sensor, calculate a detection indicator of an abnormal value in the measurement data group based on the detection signals from the green light sensor, the red light sensor, and the infrared light sensor, display the measurement data group and the detection indicator on the display unit on the same time axis, display a predetermined threshold related to the detection indicator on the display unit, and display a range where the detection indicator exceeds the threshold and a range where the detection indicator falls below the threshold in the measurement data group displayed on the display unit in different display formats.

A pulse oximeter according to a third aspect of the disclosure includes: a green light sensor configured to irradiate a subject with green light and detect the green light transmitted through or reflected from the subject; a red light sensor configured to irradiate the subject with red light and detect the red light transmitted through or reflected from the subject; an infrared light sensor configured to irradiate the subject with infrared light and detect the infrared light transmitted through or reflected from the subject; a control device configured to process detection signals from the green light sensor, the red light sensor and the infrared light sensor; and a display unit configured to display information obtained by the control device, in which the control device is configured to generate a measurement data group indicating an oxygen saturation of the subject based on the detection signals from the red light sensor and the infrared light sensor, calculate a detection indicator of an abnormal value in the measurement data group based on the detection signals from the green light sensor, the red light sensor, and the infrared light sensor, and display the measurement data group on the display unit and issue an alert regarding a posture of the subject when the detection indicator exceeds a predetermined threshold.

### BRIEF DESCRIPTION OF THE DRAWINGS

FIG. 1 is a schematic diagram showing a pulse oximeter according to an embodiment of the disclosure.

FIG. 2 is a schematic view showing a use state of the pulse oximeter according to the embodiment.

FIG. 3 is a block diagram showing a control device of the pulse oximeter according to the embodiment.

FIG. 4 is a graph showing a detection signal in the embodiment.

FIG. 5 is a graph showing an oxygen saturation and a pulse wave signal intensity ratio between colors in a normal state in the embodiment.

FIG. 6 is a graph showing the oxygen saturation, a green-red signal ratio, and a green-infrared signal ratio in the normal state in the embodiment.

FIG. 7 is a graph showing an oxygen saturation and a pulse wave signal intensity ratio between colors in an abnormal state in the embodiment.

FIG. 8 is a graph showing the oxygen saturation, the green-red signal ratio, and the green-infrared signal ratio in the abnormal state in the embodiment.

FIG. 9 is a graph showing a corrected data group from which an abnormal value is deleted in the embodiment.

FIG. 10 is a graph showing the corrected data group obtained by interpolating a deleted portion of the abnormal value in the embodiment.

FIG. 11 is a schematic diagram showing a display screen in the embodiment.

FIG. 12 is a flowchart showing processing in the embodiment.

FIG. 13 is a schematic diagram showing another embodiment of the disclosure.

### DESCRIPTION OF EMBODIMENTS

An embodiment of the disclosure will be described below.

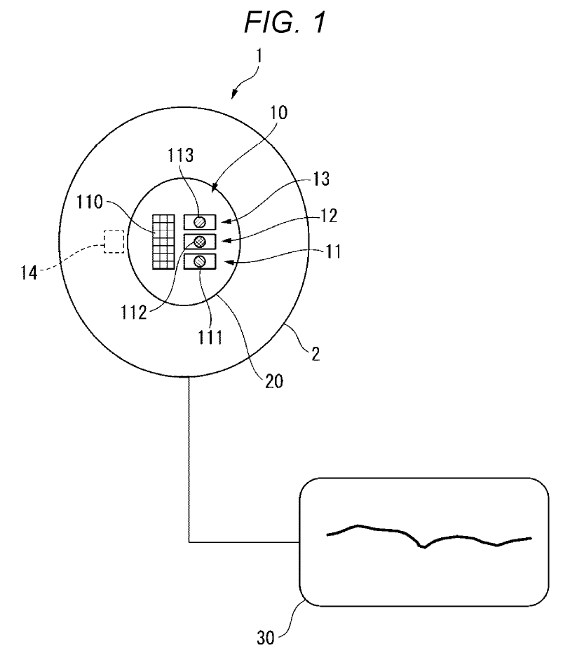

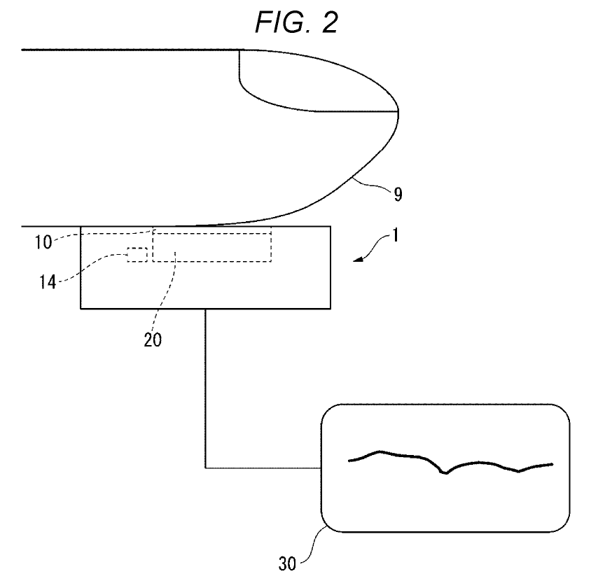

In FIGS. 1 and 2, a pulse oximeter 1 includes a sensor unit 10 and a control device 20. The sensor unit 10 and the control device 20 are accommodated in a case 2, and a display unit 30 is connected to the control device 20.

The sensor unit 10 is disposed on a surface of the case 2 and is held in close contact with a subject 9 such as a finger of a user during measurement.

A green light source 111, a red light source 112, an infrared light source 113, and an optical detector 110 are disposed in the sensor unit 10.

The green light source 111, the red light source 112, and the infrared light source 113 are, for example, light emitting diodes (LEDs) or laser diodes. It is desirable that an emission wavelength of the green light source 111 is 500 nm to 600 nm, an emission wavelength of the red light source 112 is 600 nm to 800 nm, and an emission wavelength of the infrared light source 113 is 800 nm to 1,000 nm.

The optical detector 110 is, for example, a silicon photodiode. Surfaces of the green light source 111, the red light source 112, the infrared light source 113, and the optical detector 110 of the sensor unit 10 are covered with a cover (not shown) made of a transparent resin-molded plate, such as acrylic or polycarbonate.

In the sensor unit 10, the green light source 111, the red light source 112, and the infrared light source 113 sequentially emit light under control of the control device 20, and light beams from the respective light sources are reflected by subcutaneous tissue of the subject 9 and returned to the optical detector 110. The control device 20 acquires detection signals from the optical detector 110 as a detection signal of green light, a detection signal of red light, and a detection signal of infrared light from a light emission timing of each light emitting element. The green light source 111 and the optical detector 110 constitute a green light sensor 11, the red light source 112 and the optical detector 110 constitute a red light sensor 12, and the infrared light source 113 and the optical detector 110 constitute an infrared light sensor 13.

The sensor unit 10 further includes an acceleration sensor 14 provided in the case 2.

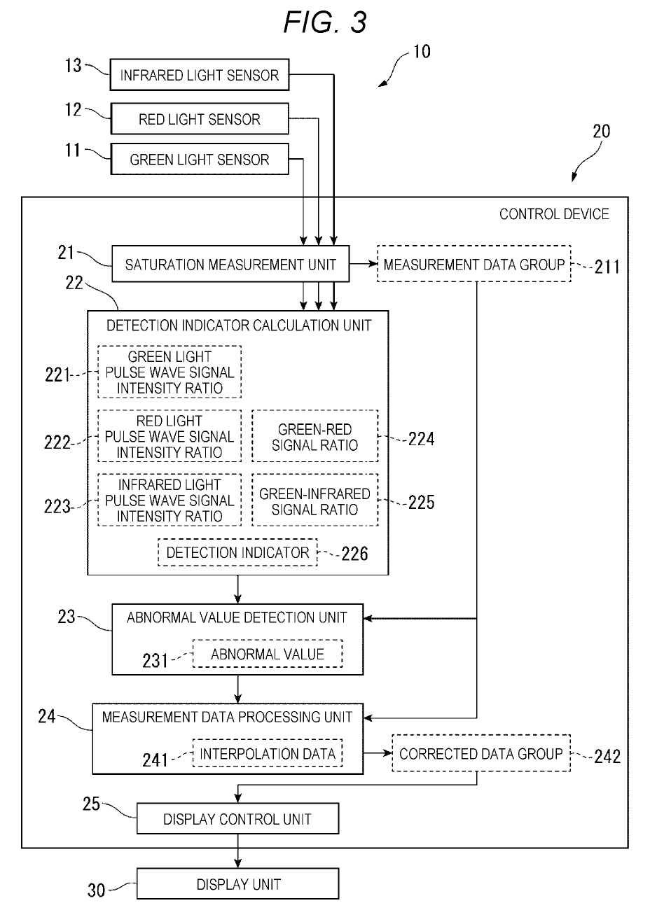

The control device 20 is implemented by a small computer system and includes a memory that stores various data and a processor that implements a desired function (see FIG. 3) by executing a program stored in the memory.

In the case 2, a battery serving as a power supply of the sensor unit 10 and the control device 20 is stored, and an input and output terminal or the like of the control device 20 is provided (not shown).

The display unit 30 is connected to the control device 20 by a wired or wireless signal unit, and can display a measurement result or the like from the control device 20. As the display unit 30, for example, in addition to a general-purpose image display panel, a portable information terminal such as a so-called smartphone can be used.

In FIG. 3, the control device 20 functions as a saturation measurement unit 21, a detection indicator calculation unit 22, an abnormal value detection unit 23, a measurement data processing unit 24, and a display control unit 25 by the processor executing a program.

The saturation measurement unit 21 controls the green light sensor 11, the red light sensor 12, and the infrared light sensor 13 of the sensor unit 10 connected to the control device 20, and acquires (receives) a detection signal.

The saturation measurement unit 21 measures a blood oxygen saturation of the subject 9 based on detection signals from the red light sensor 12 and the infrared light sensor 13, and records the blood oxygen saturation in the memory as a measurement data group 211. The detection signals from the red light sensor 12 and the infrared light sensor 13 are photoplethysmographic signals based on red light and infrared light transmitted through the subcutaneous tissue of the subject 9, and the saturation measurement unit 21 measures the oxygen saturation based on red light and infrared light pulse wave signals and records the oxygen saturation in the memory as the measurement data group 211.

The detection signal from the green light sensor 11, the detection signal from the red light sensor 12, and the detection signal from the infrared light sensor 13, which are the same as those of the saturation measurement unit 21, are connected to the detection indicator calculation unit 22.

The detection indicator calculation unit 22 calculates pulse wave signal intensity ratios (221, 222, and 223) for each of the detection signals from the green light sensor 11, the red light sensor 12, and the infrared light sensor 13.

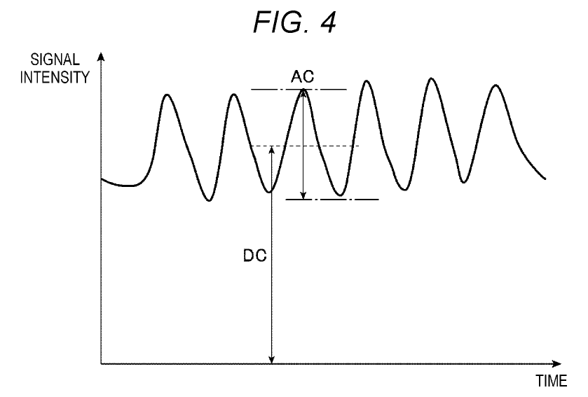

In FIG. 4, the detection signal of each color is a pulse wave where a vibration component is superimposed on a DC component, an amplitude signal intensity AC is detected from an amplitude of the vibration component, and a DC signal intensity DC is detected at the center of the amplitude. When the amplitude signal intensity AC and the DC signal intensity DC are obtained, a pulse wave signal intensity ratio of the detection signal is obtained by a ratio AC/DC of the amplitude signal intensity AC to the DC signal intensity DC.

Accordingly, the green light pulse wave signal intensity ratio 221 that is a pulse wave signal intensity ratio (ratio AC/DC) of the detection signal from the green light sensor 11, the red light pulse wave signal intensity ratio 222 that is a pulse wave signal intensity ratio of the detection signal from the red light sensor 12, and the infrared light pulse wave signal intensity ratio 223 that is a pulse wave signal intensity ratio of the detection signal from the infrared light sensor 13 are calculated.

The detection indicator calculation unit 22 calculates, using the obtained pulse wave signal intensity ratios of the respective colors, a green-red signal ratio 224 that is a ratio between the green light pulse wave signal intensity ratio 221 and the red light pulse wave signal intensity ratio 222, and a green-infrared signal ratio 225 that is a ratio between the green light pulse wave signal intensity ratio 221 and the infrared light pulse wave signal intensity ratio 223.

For the green-red signal ratio 224, a ratio Rrg = (ACr/DCr)/(ACg/DCg) of a red light pulse wave signal intensity ratio ACr/DCr to a green light pulse wave signal intensity ratio ACg/DCg is calculated.

For the green-infrared signal ratio 225, a ratio Rig = (ACi/DCi)/(ACg/DCg) of an infrared light pulse wave signal intensity ratio ACi/DCi to the green light pulse wave signal intensity ratio ACg/DCg is calculated.

The detection indicator calculation unit 22 sets at least one of the green-red signal ratio 224 and the green-infrared signal ratio 225 as a detection indicator 226. The detection indicator 226 may be a designated one of the green-red signal ratio 224 and the green-infrared signal ratio 225, a larger or smaller one thereof, or a sum or an average value of both.

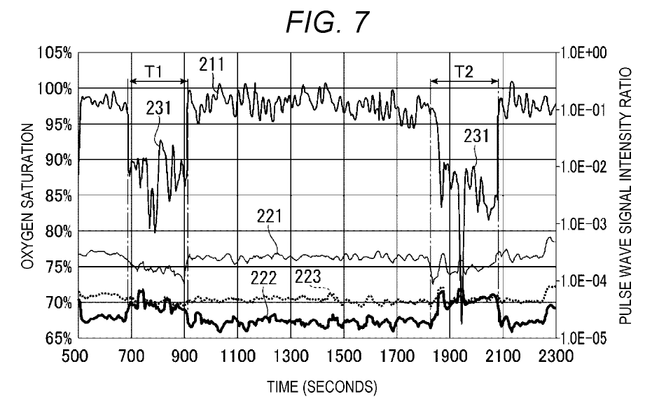

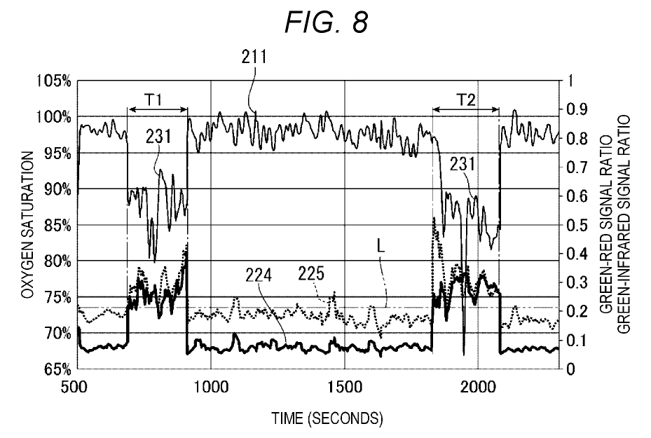

The abnormal value detection unit 23 detects an abnormal value 231 (see FIGS. 7 and 8) appearing in the measurement data group 211 based on the detection indicator 226.

The abnormal value detection unit 23 refers to a change over time in the detection indicator 226, detects a data variation occurring when the subject 9 is in a specific posture, and determines that an anomaly occurs in the oxygen saturation measurement by the saturation measurement unit 21. Then, the measurement data group 211 in a period in which it is determined that the anomaly occurs is detected as the abnormal value 231.

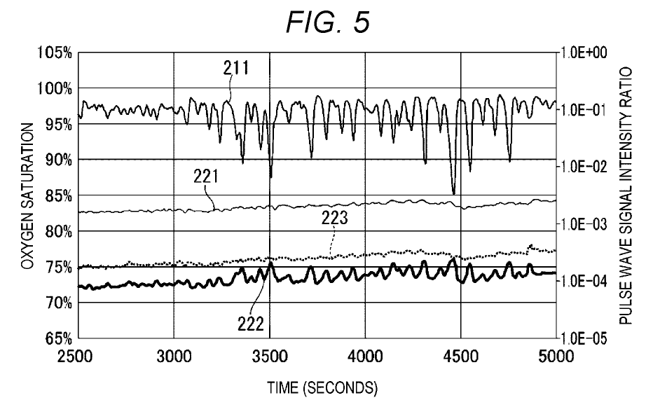

A detection operation will be described in detail later with reference to FIGS. 5 to 8.

The measurement data processing unit 24 deletes, from the measurement data group 211, the abnormal value 231 detected by the abnormal value detection unit 23, and connects the deleted portion with interpolation data 241 to create a corrected data group 242. Any data interpolation method such as linear interpolation or spline interpolation can be used for data interpolation processing.

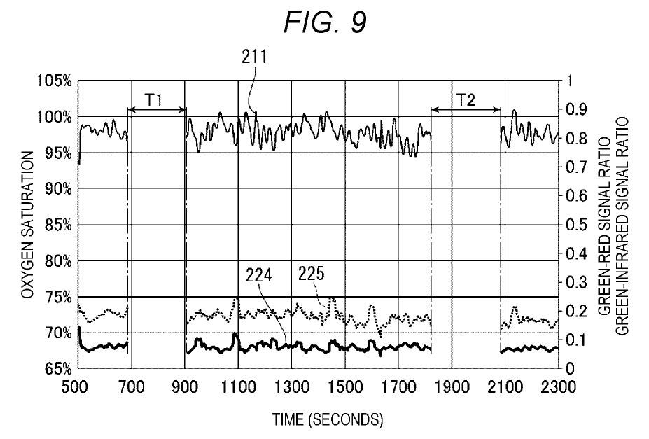

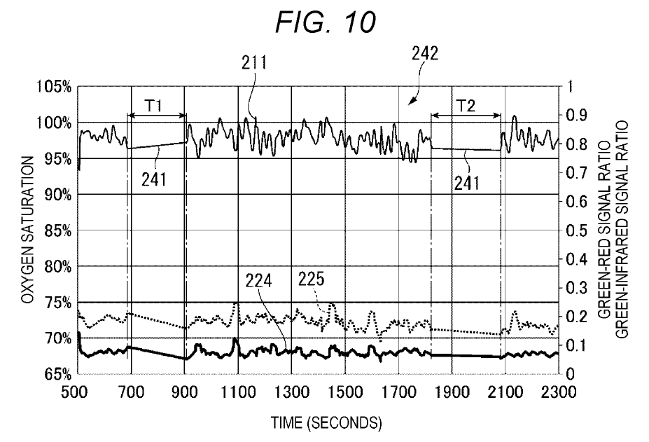

The processing by the measurement data processing unit 24 will be described later in detail with reference to FIGS. 9 and 10.

The display control unit 25 displays various data obtained by the control device 20 on the display unit 30.

As display contents, in addition to the corrected data group 242 obtained by the measurement data processing unit 24, the original measurement data group 211 obtained by the saturation measurement unit 21 and the detection indicator 226 (the green-red signal ratio 224 or the green-infrared signal ratio 225) may be displayed, and an alert display or the like based on these may be displayed. Further, an interval where the oxygen saturation decreases over a predetermined time (intervals T1, T2, and the like) or the number of times the oxygen saturation decreases may be calculated based on the detection indicator 226, and a calculation result may be displayed on the display unit 30.

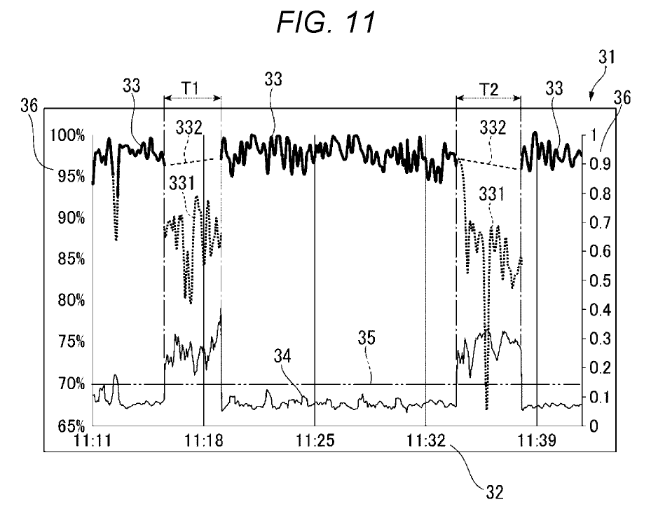

The display by the display control unit 25 will be described in detail later with reference to FIG. 11.

### Abnormal Value Detection

Next, abnormal value detection by the detection indicator calculation unit 22 will be described in more detail.

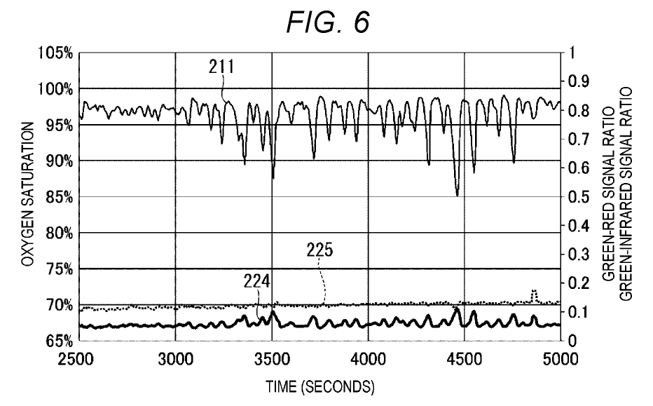

FIGS. 5 and 6 show the measurement data group 211 of the oxygen saturation, the pulse wave signal intensity ratios (221, 222, and 223) of light of the respective colors, the green-red signal ratio 224, and the green-infrared signal ratio 225 in a normal state.

In FIG. 5, the measurement data group 211 is oxygen saturation data acquired by the saturation measurement unit 21, and the oxygen saturation decreases irregularly but is basically in a consistent numerical value range.

The green light pulse wave signal intensity ratio 221 and the infrared light pulse wave signal intensity ratio 223 both show a right upward trend and have small data variations. The red light pulse wave signal intensity ratio 222 shows a variation correlated with a decrease in the oxygen saturation in the measurement data group 211 while showing a right upward trend.

In FIG. 6, in the green-red signal ratio 224, the right upward trends observed in the green light pulse wave signal intensity ratio 221 and the red light pulse wave signal intensity ratio 222 are mitigated and a variation due to the decrease in the oxygen saturation is also reduced. In the green-infrared signal ratio 225, the right upward trend and the variation observed in the infrared light pulse wave signal intensity ratio 223 are mitigated.

In this way, in the normal state, each piece of data indicates a substantially continuous value, and no significant variation is observed.

FIGS. 7 and 8 show the measurement data group 211 of the oxygen saturation, the pulse wave signal intensity ratios (221, 222, and 223) of light of the respective colors, the green-red signal ratio 224, and the green-infrared signal ratio 225 in an abnormal state.

In FIG. 7, the measurement data group 211 is basically in a consistent numerical value range, but has the abnormal value 231 whose numerical value significantly decreases in the interval T1 and the interval T2.

The green light pulse wave signal intensity ratio 221 exhibits a numerical value slightly lower than usual in the interval T1 and the interval T2.

The red light pulse wave signal intensity ratio 222 exhibits a numerical value significantly higher than usual in the interval T1 and the interval T2.

The infrared light pulse wave signal intensity ratio 223 does not change in the interval T1 and the interval T2.

A cause of occurrence of the abnormal value 231 in the measurement data group 211 includes a situation where a blood flow to a detection site is compressed due to a posture change of the subject 9 and a blood flow rate temporarily decreases.

The blood flow of the subject may also be compressed in a state where a detection target of the green light sensor 11, the red light sensor 12, and the infrared light sensor 13 is a limb of the subject 9, and the limb that is the detection target of the subject 9 in a supine position hangs downward below torso of the subject 9, or a state where the limb that is the detection target of the subject 9 in a supine position is pinned under another part of the subject 9.

Alternatively, the blood flow of the subject may also be compressed in a state where the detection target of the green light sensor 11, the red light sensor 12, and the infrared light sensor 13 is a head of the subject 9 and a blood vessel of a neck of the subject 9 is compressed.

The abnormal value 231 due to such a cause tends to continuously appear unlike the decrease in the oxygen saturation to be originally measured.

In FIG. 8, in the green-red signal ratio 224 and the green-infrared signal ratio 225, numerical values increase more significantly than usual in the interval T1 and the interval T2. In particular, in the interval T1 and the interval T2, the green light pulse wave signal intensity ratio 221 is low, the red light pulse wave signal intensity ratio 222 is high, both ratios change, and thus a numerical value change in the green-red signal ratio 224 at an interval end is more clear.

In this way, in the abnormal state, a clear change is observed in the green-red signal ratio 224 or the green-infrared signal ratio 225 used as the detection indicator 226. Therefore, the detection indicator calculation unit 22 can specify the intervals T1 and T2 where the abnormal value 231 occurs by detecting the green-red signal ratio 224 or the green-infrared signal ratio 225.

The detection indicator calculation unit 22 can use a predetermined threshold L to specify the intervals T1 and T2. When the green-red signal ratio 224 or the green-infrared signal ratio 225 is used as the detection indicator 226, the threshold L larger than each value in the normal state is set, and the abnormal value 231 can be determined when the threshold L is exceeded.

### Data Processing and Display Processing

Next, contents of data processing by the measurement data processing unit 24 and display control by the display control unit 25 will be described in more detail.

FIGS. 9 and 10 show deletion or interpolation processing of the abnormal value 231 by the measurement data processing unit 24.

In FIG. 9, the measurement data processing unit 24 deletes data in the intervals T1 and T2 in the measurement data group 211 based on the abnormal value 231 detected by the abnormal value detection unit 23.

In FIG. 10, the measurement data processing unit 24 interpolates the intervals T1 and T2 from which the data is deleted in the measurement data group 211 with the interpolation data 241, and sets the measurement data group 211 interpolated with the interpolation data 241 as the corrected data group 242.

FIG. 11 shows a display screen 31 displayed on the display unit 30 by the display control unit 25.

The display screen 31 displays, along a time axis 32 displayed at a lower part, an oxygen saturation graph 33 indicating the corrected data group 242 generated by the measurement data processing unit 24, a detection indicator graph 34 indicating the detection indicator 226 (the green-red signal ratio 224 or the green-infrared signal ratio 225) used by the abnormal value detection unit 23, and a threshold display 35 indicating the threshold L for determining the abnormal value 231.

In the oxygen saturation graph 33, an abnormal value graph 331 indicating the abnormal value 231 and an interpolation data graph 332 indicating the interpolation data 241 may be displayed in the intervals T1 and T2. On both sides of the display screen 31, vertical axis displays 36 indicating numerical values of the oxygen saturation graph 33 and the detection indicator graph 34 are displayed.

The oxygen saturation graph 33, the detection indicator graph 34, the abnormal value graph 331, the interpolation data graph 332, and the vertical axis displays 36 are displayed in different display formats that are easily distinguished, for example, display formats having different saturation, brightness, transparency, or line types, or a combination thereof.

In particular, in the oxygen saturation graph 33, the intervals T1 and T2, that is, a range where the detection indicator 226 exceeds the threshold L and a range where the detection indicator 226 falls below the threshold L other than the intervals T1 and T2 are displayed in display formats different from each other, for example, display formats having different saturation, brightness, transparency, or line types, or a combination thereof. Among these, the abnormal value graph 331 and the interpolation data graph 332 displayed in the intervals T1 and T2 may be displayed in gray or the like to be less distinguishable than other portions. The threshold display 35 indicating the threshold L is not limited to a straight line, and may be displayed by a mark or a shape added to a part of the display of the detection indicator 226.

As display contents of the display screen 31, the original measurement data group 211 may be displayed in addition to the corrected data group 242, and an alert display regarding the posture of the subject 9 may be displayed based on the detection indicator 226 (the green-red signal ratio 224 or the green-infrared signal ratio 225). The alert display may be an alarm by voice. The data (abnormal value graph 331) determined as the abnormal value 231 in the measurement data group 211 is excluded from the corrected data group 242 and may be excluded from an alert target.

Further, the interval where the oxygen saturation decreases over a predetermined time (the intervals T1, T2, and the like) or the number of times the oxygen saturation decreases may be calculated based on the detection indicator 226, and a calculation result may be displayed on the display unit 30.

### Operation of Pulse Oximeter

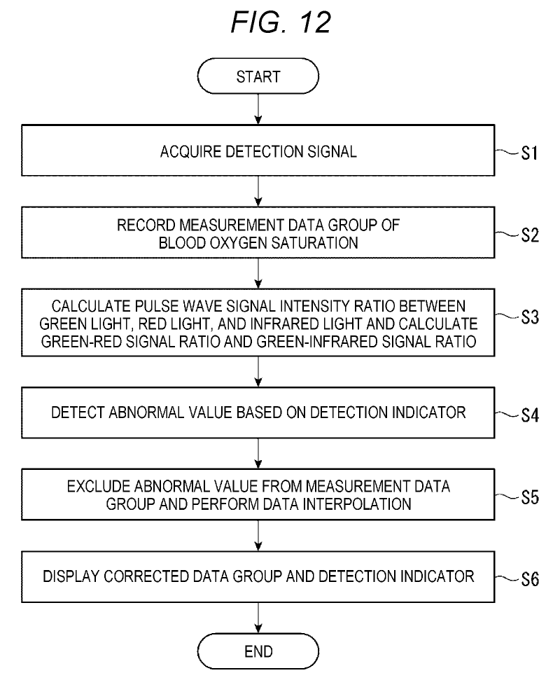

FIG. 12 shows an overview of processing executed by the pulse oximeter 1 of the embodiment.

In the pulse oximeter 1, when the processor of the control device 20 executes a program, the following processing is executed by each unit of the sensor unit 10 and the control device 20.

In processing S1, the sensor unit 10 irradiates the subject 9 with green light, red light and infrared light and detects the green light, the red light, and the infrared light transmitted through or reflected from the subject 9.

In processing S2, the saturation measurement unit 21 measures the oxygen saturation of the subject 9 based on the detection signals of the red light and the infrared light, and records the oxygen saturation as the measurement data group 211 (see FIGS. 5 and 6).

In processing S3, the detection indicator calculation unit 22 calculates the pulse wave signal intensity ratios (221, 222, and 223) of light of the respective colors based on the detection signals of the green light, the red light, and the infrared light, further calculates the green-red signal ratio 224 and the green-infrared signal ratio 225, and sets one thereof as the detection indicator 226.

In processing S4, the abnormal value detection unit 23 detects the abnormal value 231 appearing in the measurement data group 211 based on the detection indicator 226 (see FIGS. 7 and 8).

In processing S5, the measurement data processing unit 24 removes data of the abnormal value 231 from the measurement data group 211 (see FIG. 9), and interpolates a portion from which the data is removed with the interpolation data 241 to generate the corrected data group 242 (see FIG. 10).

In processing S6, the display control unit 25 displays the corrected data group 242 and the detection indicator 226 on the display unit 30 (see FIG. 11).

In the pulse oximeter 1 of the embodiment, it is possible to check whether a function is normal by the following operation.

A film or sheet that can prevent transmission of light having an inspection wavelength of the green light sensor 11, the red light sensor 12, and the infrared light sensor 13 is prepared, and measurement is performed in a state where the film or sheet is interposed between the subject 9 and the sensor unit 10 of the pulse oximeter 1.

When the pulse oximeter 1 detects the green-red signal ratio 224, a function of detecting a posture anomaly of the subject 9 is impaired when the film preventing the red light is interposed, and the function is recovered by removing the film.

When the pulse oximeter 1 detects the green-infrared signal ratio 225, the function of detecting the posture anomaly of the subject 9 is impaired when the film preventing the infrared light is interposed, and the function is recovered by removing the film.

When the film preventing the green light is interposed, the function of detecting the posture anomaly of the subject 9 is impaired regardless of whether the pulse oximeter 1 uses the green-red signal ratio 224 or the green-infrared signal ratio 225, and the function is recovered by removing the film.

In this way, the function of the pulse oximeter 1 can be determined by blocking the inspection light.

### Functions and Effects of Embodiment

The pulse oximeter 1 of the embodiment includes the green light sensor 11 that irradiates the subject 9 with green light and detects the green light transmitted through or reflected from the subject 9, the red light sensor 12 that irradiates the subject 9 with red light and detects the red light transmitted through or reflected from the subject 9, the infrared light sensor 13 that irradiates the subject 9 with infrared light and detects the infrared light transmitted through or reflected from the subject 9, and the control device 20 that processes the detection signals from the green light sensor 11, the red light sensor 12, and the infrared light sensor 13. The control device 20 includes the saturation measurement unit 21 that generates the measurement data group 211 indicating the oxygen saturation of the subject 9 based on the detection signals from the red light sensor 12 and the infrared light sensor 13, the detection indicator calculation unit 22 that calculates the ratio between the detection signals from the green light sensor 11, the red light sensor 12, and the infrared light sensor 13 as the detection indicator 226, the abnormal value detection unit 23 that detects the abnormal value 231 of the oxygen saturation based on the detection indicator 226. The detection indicator calculation unit 22 calculates, as the detection indicator 226, at least one of the green-red signal ratio 224 that is the ratio between the green light pulse wave signal intensity ratio 221, which indicates the ratio of the amplitude of the vibration component in the detection signal output by the green light sensor 11 to the signal intensity of the detection signal, and the red light pulse wave signal intensity ratio 222, which indicates the ratio of the amplitude of the vibration component in the detection signal output by the red light sensor 12 to the signal intensity of the detection signal, and the green-infrared signal ratio 225 that is the ratio between the green light pulse wave signal intensity ratio 221 and the infrared light pulse wave signal intensity ratio 223, which indicates the ratio of the amplitude of the vibration component in the detection signal output by the infrared light sensor 13 to the signal intensity of the detection signal.

In such a pulse oximeter 1, the measurement data group 211 of the blood oxygen saturation of the subject 9 is obtained by the red light sensor 12, the infrared light sensor 13, and the saturation measurement unit 21 of the control device 20. The detection indicator 226 is calculated by the detection indicator calculation unit 22. Since the abnormal value detection unit 23 detects the abnormal value 231 for the measurement data group 211 based on the detection indicator 226, a measurement error caused by a body motion of the subject 9 can be removed to improve measurement accuracy.

The detection indicator 226 is calculated based on the ratio between the detection signals from the green light sensor 11, the red light sensor 12, and the infrared light sensor 13. In the processing of the detection signal from each color light sensor (11, 12, and 13), using the pulse wave signal intensity ratio of light of the respective colors (221, 222, and 223), that is, the ratio of the amplitude of the vibration component in the detection signal output by each sensor to the signal intensity of the detection signal, a rate of change in the vibration component of each detection signal is relativized, and a numerical value suitable for comparison can be obtained.

As the detection indicator 226, at least one of the green-red signal ratio 224 that is the ratio between the green light pulse wave signal intensity ratio 221 and the red light pulse wave signal intensity ratio 222, and the green-infrared signal ratio 225 that is the ratio between the green light pulse wave signal intensity ratio 221 and the infrared light pulse wave signal intensity ratio 223 is used. Green light commonly used for the green-red signal ratio 224 and the green-infrared signal ratio 225 is easily absorbed by blood hemoglobin, and the detection signal from the green light sensor 11 indicates an amount of blood hemoglobin. During sleep, shallow breathing caused by sleep apnea syndrome leads to a decrease in the oxygen saturation. Even when the oxygen saturation decreases due to the sleep apnea syndrome, the detection signal from the green light sensor 11 does not change unless a blood flow changes. On the other hand, when the blood flow is restricted due to a posture change of the subject 9, the detection signal from the green light sensor 11 decreases as a hemoglobin flow rate decreases. Therefore, using at least one of the green-red signal ratio 224 and the green-infrared signal ratio 225 as the detection indicator 226, the abnormal value caused by the body motion of the subject can be detected.

The pulse oximeter 1 of the embodiment includes the green light sensor 11 that irradiates the subject 9 with green light and detects the green light transmitted through or reflected from the subject 9, the red light sensor 12 that irradiates the subject 9 with red light and detects the red light transmitted through or reflected from the subject 9, the infrared light sensor 13 that irradiates the subject 9 with infrared light and detects the infrared light transmitted through or reflected from the subject, the control device 20 that processes the detection signals from the green light sensor 11, the red light sensor 12, and the infrared light sensor 13, and the display unit 30 that displays information obtained by the control device 20. The control device 20 generates the measurement data group 211 indicating the oxygen saturation of the subject 9 based on the detection signals from the red light sensor 12 and the infrared light sensor 13, calculates the detection indicator 226 of the abnormal value 231 in the measurement data group 211 based on the detection signals from the green light sensor 11, the red light sensor 12, and the infrared light sensor 13, displays the measurement data group 211 and the detection indicator 226 on the display unit 30 on the same time axis, displays the predetermined threshold L related to the detection indicator 226 on the display unit 30, and displays the range where the detection indicator 226 exceeds the threshold L and the range where the detection indicator 226 falls below the threshold L in the measurement data group 211 displayed on the display unit 30 in different display formats.

In such a pulse oximeter 1, the measurement data group 211 of the blood oxygen saturation of the subject 9 is obtained by the red light sensor 12, the infrared light sensor 13, and the control device 20. The control device 20 displays the detection indicator 226 indicating the abnormal value 231 together with the measurement data group 211 on the display unit 30, particularly displays the range where the detection indicator 226 exceeds the threshold L and the range where the detection indicator 226 falls below the threshold L in the measurement data group 211 displayed on the display unit 30 in different display formats, and thus the user can remove a measurement error caused by a body motion of the subject to improve measurement accuracy.

### Other Embodiments

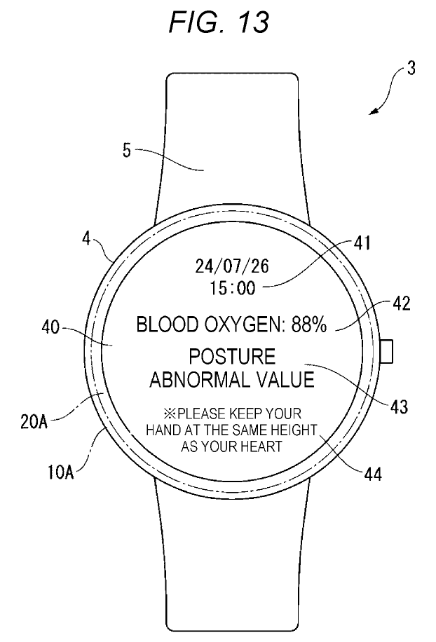

FIG. 13 shows another embodiment of the disclosure.

In FIG. 13, a pulse oximeter 3 has a flat disk-shaped case 4 and a belt 5, and can be worn on a wrist of the user.

A display unit 40 is formed at a surface of the case 4, and a current time display 41, an oxygen saturation display 42, a main alert display 43, and an alert detail display 44 are displayed on the display unit 40. A control device 20A is stored inside the case 4, and a sensor unit 10A is formed at a back side of the case 4.

The sensor unit 10A includes the green light sensor 11, the red light sensor 12, and the infrared light sensor 13 similarly to the sensor unit 10 of the embodiment described above. The green light sensor 11, the red light sensor 12, and the infrared light sensor 13 are disposed to be in close contact with a surface of the wrist of the user when the pulse oximeter 3 is attached to the wrist of the user with the belt 5, and the surface of the wrist serves as the subject 9.

The control device 20A is implemented similarly to the control device 20 described above, measures the blood oxygen saturation of the subject 9 based on the detection signals from the green light sensor 11, the red light sensor 12, and the infrared light sensor 13 to generate the measurement data group 211, and calculates the detection indicator 226 for the measurement data group 211 to detect the abnormal value 231.

The control device 20A displays the oxygen saturation data (including the abnormal value 231) at the current time or any time in the past of the measurement data group 211 on the oxygen saturation display 42, and displays the main alert display 43 and the alert detail display 44 according to the detection indicator 226 of the same time based on an operation of the user.

For example, in a state where the detection indicator 226 at the displayed time of the measurement data group 211 indicates the abnormal value 231, a highly distinguishable character string such as "POSTURE ABNORMAL VALUE" is displayed as the main alert display 43, and specific guidance such as "PLEASE KEEP YOUR HAND AT THE SAME HEIGHT AS YOUR HEART" is displayed as the alert detail display 44. In a state where the detection indicator 226 is in a normal range, a character string such as "NORMAL VALUE" may be displayed on the main alert display 43, or a caution message such as "SLIGHTLY LOW VALUE" may be displayed according to a data numerical value of the oxygen saturation display 42.

In such a pulse oximeter 3, the red light sensor 12, the infrared light sensor 13, and the control device 20A can measure the blood oxygen saturation of the subject 9 and display the measurement result on the display unit 40.

In the control device 20A, the abnormal value 231 of the measurement data group 211 can be detected based on the detection indicator 226 obtained from the green light sensor 11, the red light sensor 12, and the infrared light sensor 13, and a warning message or the like can be displayed on the main alert display 43 and the alert detail display 44 according to the detection indicator 226.

The pulse oximeter 3 of the embodiment includes, as the sensor unit 10A, the green light sensor 11 that irradiates the subject 9 with green light and detects the green light transmitted through or reflected from the subject 9, the red light sensor 12 that irradiates the subject 9 with red light and detects the red light transmitted through or reflected from the subject 9, and the infrared light sensor 13 that irradiates the subject 9 with infrared light and detects the infrared light transmitted through or reflected from the subject 9, the control device 20A that processes the detection signals from the green light sensor 11, the red light sensor 12, and the infrared light sensor 13, and the display unit 40 that displays information obtained by the control device 20A. The control device 20A generates the measurement data group 211 indicating the oxygen saturation of the subject 9 based on the detection signals from the red light sensor 12 and the infrared light sensor 13, calculates the detection indicator 226 of the abnormal value 231 in the measurement data group 211 based on the detection signals from the green light sensor 11, the red light sensor 12, and the infrared light sensor 13, and displays the measurement data group 211 on the display unit 40 and issues an alert (the main alert display 43 and the alert detail display 44) regarding the posture of the subject 9 when the detection indicator 226 exceeds the predetermined threshold L.

In such a pulse oximeter 3, the measurement data group 211 of the blood oxygen saturation of the subject 9 is obtained by the red light sensor 12, the infrared light sensor 13, and the control device 20A. The control device 20A displays the detection indicator 226 indicating the abnormal value 231 together with the measurement data group 211 on the display unit 30, particularly issues the alert regarding the posture of the subject 9 when the detection indicator 226 exceeds the predetermined threshold L, and thus the user can remove a measurement error caused by a body motion of the subject to improve measurement accuracy.

### Modifications

The disclosure is not limited to the embodiments described above, and modifications and the like within a range where the object of the disclosure can be obtained are contained in the disclosure.

The disclosure is not limited to the reflective pulse oximeter described in the embodiments, and can also be applied to a transmissive pulse oximeter.

In the present specification, the pulse oximeter that measures the blood oxygen saturation has been described, and it is needless to say that the disclosure can be used not only in a device called a pulse oximeter but also in a blood oxygen wellness apparatus that displays a measurement result as a "blood oxygen level". It should be understood that the pulse oximeter includes the blood oxygen wellness apparatus in this specification.

### Summary of Disclosure

A pulse oximeter according to a first aspect of the disclosure includes: a green light sensor configured to irradiate a subject with green light and detect the green light transmitted through or reflected from the subject; a red light sensor configured to irradiate the subject with red light and detect the red light transmitted through or reflected from the subject; an infrared light sensor configured to irradiate the subject with infrared light and detect the infrared light transmitted through or reflected from the subject; and a control device configured to process detection signals from the green light sensor, the red light sensor, and the infrared light sensor, in which the control device includes a saturation measurement unit that generates a measurement data group indicating an oxygen saturation of the subject based on the detection signals from the red light sensor and the infrared light sensor, a detection indicator calculation unit that calculates, as a detection indicator, a ratio between the detection signals from the green light sensor, the red light sensor, and the infrared light sensor, and an abnormal value detection unit that detects an abnormal value of the oxygen saturation based on the detection indicator, and the detection indicator calculation unit calculates, as the detection indicator, at least one of a green-red signal ratio that is a ratio between a green light pulse wave signal intensity ratio, which indicates a ratio of an amplitude of a vibration component in the detection signal output by the green light sensor to a signal intensity of the detection signal, and a red light pulse wave signal intensity ratio, which indicates a ratio of an amplitude of a vibration component in the detection signal output by the red light sensor to a signal intensity of the detection signal, and a green-infrared signal ratio that is a ratio between the green light pulse wave signal intensity ratio and an infrared light pulse wave signal intensity ratio, which indicates a ratio of an amplitude of a vibration component in the detection signal output by the infrared light sensor to a signal intensity of the detection signal.

In such a pulse oximeter, the measurement data group of the blood oxygen saturation of the subject is obtained by the red light sensor, the infrared light sensor, and the saturation measurement unit of the control device. The detection indicator is calculated by the detection indicator calculation unit. Since the abnormal value detection unit detects the abnormal value for the measurement data group based on the detection indicator, a measurement error caused by a body motion of the subject can be removed to improve measurement accuracy.

The detection indicator is calculated based on the ratio between the detection signals from the green light sensor, the red light sensor, and the infrared light sensor. In the processing of the detection signal from each color light sensor, using the pulse wave signal intensity ratio of light of the respective colors, that is, the ratio of the amplitude of the vibration component in the detection signal output by each sensor to the signal intensity of the detection signal, a rate of change in the vibration component of each detection signal is relativized, and a numerical value suitable for comparison can be obtained.

As the detection indicator, at least one of the green-red signal ratio that is the ratio between the green light pulse wave signal intensity ratio and the red light pulse wave signal intensity ratio, and the green-infrared signal ratio that is the ratio between the green light pulse wave signal intensity ratio and the infrared light pulse wave signal intensity ratio is used. Green light commonly used for the green-red signal ratio and the green-infrared signal ratio is easily absorbed by blood hemoglobin, and the detection signal from the green light sensor indicates an amount of blood hemoglobin. During sleep, shallow breathing caused by sleep apnea syndrome leads to a decrease in the oxygen saturation. Even when the oxygen saturation decreases due to the sleep apnea syndrome, the detection signal from the green light sensor does not change unless a blood flow changes. On the other hand, when the blood flow is restricted due to a posture change of the subject, the detection signal from the green light sensor decreases as a hemoglobin flow rate decreases. Therefore, using at least one of the green-red signal ratio and the green-infrared signal ratio as the detection indicator, the abnormal value caused by the body motion of the subject can be detected.

In the pulse oximeter according to the first aspect of the disclosure, it is preferable that the abnormal value detection unit determines that the abnormal value appears when the detection indicator corresponds to a state where a blood flow of the subject is compressed.

In the pulse oximeter according to the first aspect of the disclosure, it is preferable that a detection target of the green light sensor, the red light sensor, and the infrared light sensor is a limb of the subject, and the state where the blood flow of the subject is compressed is a state where the limb that is the detection target of the subject in a supine position hangs downward below torso of the subject, or a state where the limb that is the detection target of the subject in a supine position is pinned under another part of the subject.

In the pulse oximeter according to the first aspect of the disclosure, it is preferable that a detection target of the green light sensor, the red light sensor, and the infrared light sensor is a head of the subject, and the state where the blood flow of the subject is compressed is a state where a blood vessel of a neck of the subject is compressed.

In the pulse oximeter according to the first aspect of the disclosure, it is preferable that the abnormal value detection unit determines that the abnormal value appears when the detection indicator exceeds a predetermined threshold.

In the pulse oximeter according to the first aspect of the disclosure, it is preferable that the control device further includes a measurement data processing unit, and the measurement data processing unit deletes data determined to be the abnormal value from the measurement data group.

In the pulse oximeter according to the first aspect of the disclosure, it is preferable that the control device further includes an alert issuing unit that issues an oxygen saturation decrease alert based on the measurement data group, and the alert issuing unit excludes data determined to be the abnormal value from an alert target.

A pulse oximeter according to a second aspect of the disclosure includes: a green light sensor configured to irradiate a subject with green light and detect the green light transmitted through or reflected from the subject; a red light sensor configured to irradiate the subject with red light and detect the red light transmitted through or reflected from the subject; an infrared light sensor configured to irradiate the subject with infrared light and detect the infrared light transmitted through or reflected from the subject; a control device configured to process detection signals from the green light sensor, the red light sensor and the infrared light sensor; and a display unit configured to display information obtained by the control device, in which the control device generates a measurement data group indicating an oxygen saturation of the subject based on the detection signals from the red light sensor and the infrared light sensor, calculates a detection indicator of an abnormal value in the measurement data group based on the detection signals from the green light sensor, the red light sensor, and the infrared light sensor, displays the measurement data group and the detection indicator on the display unit on the same time axis, displays a predetermined threshold related to the detection indicator on the display unit, and displays a range where the detection indicator exceeds the threshold and a range where the detection indicator falls below the threshold in the measurement data group displayed on the display unit in different display formats.

In such a pulse oximeter, the measurement data group of the blood oxygen saturation of the subject is obtained by the red light sensor, the infrared light sensor, and the control device. The control device displays the detection indicator indicating the abnormal value together with the measurement data group on the display unit, particularly displays the range where the detection indicator exceeds the threshold and the range where the detection indicator falls below the threshold in the measurement data group displayed on the display unit in different display formats, and thus the user can remove a measurement error caused by a body motion of the subject to improve measurement accuracy.

In the pulse oximeter according to the second aspect of the disclosure, it is preferable that the control device displays the threshold by a mark or a shape added to the display of the detection indicator.

In the pulse oximeter according to the second aspect of the disclosure, it is preferable that the control device displays the range where the detection indicator exceeds the threshold and the range where the detection indicator falls below the threshold in different display formats using any one or a combination of saturation, brightness, and transparency.

In the pulse oximeter according to the second aspect of the disclosure, it is preferable that, when the detection indicator exceeds the threshold, the control device displays the measurement data group on the display unit and issues an alert regarding a posture of the subject.

A pulse oximeter according to a third aspect of the disclosure includes: a green light sensor configured to irradiate a subject with green light and detect the green light transmitted through or reflected from the subject; a red light sensor configured to irradiate the subject with red light and detect the red light transmitted through or reflected from the subject; an infrared light sensor configured to irradiate the subject with infrared light and detect the infrared light transmitted through or reflected from the subject; a control device configured to process detection signals from the green light sensor, the red light sensor and the infrared light sensor; and a display unit configured to display information obtained by the control device, in which the control device generates a measurement data group indicating an oxygen saturation of the subject based on the detection signals from the red light sensor and the infrared light sensor, calculates a detection indicator of an abnormal value in the measurement data group based on the detection signals from the green light sensor, the red light sensor, and the infrared light sensor, and displays the measurement data group on the display unit and issues an alert regarding a posture of the subject when the detection indicator exceeds a predetermined threshold.

In such a pulse oximeter, the measurement data group of the blood oxygen saturation of the subject is obtained by the red light sensor, the infrared light sensor, and the control device. The control device displays the detection indicator indicating the abnormal value together with the measurement data group on the display unit, particularly issues the alert regarding the posture of the subject when the detection indicator exceeds the predetermined threshold, and thus the user can remove a measurement error caused by a body motion of the subject to improve measurement accuracy.

What is claimed is:

1. A pulse oximeter, comprising:
a green light sensor configured to irradiate a subject with green light and detect the green light transmitted through or reflected from the subject;
a red light sensor configured to irradiate the subject with red light and detect the red light transmitted through or reflected from the subject;
an infrared light sensor configured to irradiate the subject with infrared light and detect the infrared light transmitted through or reflected from the subject; and
a control device configured to process detection signals from the green light sensor, the red light sensor, and the infrared light sensor, wherein
the control device includes
a saturation measurement unit that generates a measurement data group indicating an oxygen saturation of the subject based on the detection signals from the red light sensor and the infrared light sensor,
a detection indicator calculation unit that calculates, as a detection indicator, a ratio between the detection signals from the green light sensor, the red light sensor, and the infrared light sensor, and
an abnormal value detection unit that detects an abnormal value of the oxygen saturation based on the detection indicator, and
the detection indicator calculation unit calculates, as the detection indicator, at least one of
a green-red signal ratio that is a ratio between a green light pulse wave signal intensity ratio, which indicates a ratio of an amplitude of a vibration component in the detection signal output by the green light sensor to a signal intensity of the detection signal, and a red light pulse wave signal intensity ratio, which indicates a ratio of an amplitude of a vibration component in the detection signal output by the red light sensor to a signal intensity of the detection signal, and
a green-infrared signal ratio that is a ratio between the green light pulse wave signal intensity ratio and an infrared light pulse wave signal intensity ratio, which indicates a ratio of an amplitude of a vibration component in the detection signal output by the infrared light sensor to a signal intensity of the detection signal.

2. The pulse oximeter according to claim 1, wherein
the abnormal value detection unit determines that the abnormal value appears when the detection indicator corresponds to a state where a blood flow of the subject is compressed.

3. The pulse oximeter according to claim 2, wherein
a detection target of the green light sensor, the red light sensor, and the infrared light sensor is a limb of the subject, and
the state where the blood flow of the subject is compressed is a state where the limb that is the detection target of the subject in a supine position hangs downward below torso of the subject, or a state where the limb that is the detection target of the subject in a supine position is pinned under another part of the subject.

4. The pulse oximeter according to claim 2, wherein
a detection target of the green light sensor, the red light sensor, and the infrared light sensor is a head of the subject, and
the state where the blood flow of the subject is compressed is a state where a blood vessel of a neck of the subject is compressed.

5. The pulse oximeter according to claim 1, wherein
the abnormal value detection unit determines that the abnormal value appears when the detection indicator exceeds a predetermined threshold.

6. The pulse oximeter according to claim 5, wherein
the control device further includes a measurement data processing unit, and
the measurement data processing unit deletes data determined to be the abnormal value from the measurement data group.

7. The pulse oximeter according to claim 5, wherein
the control device is configured to issue an oxygen saturation decrease alert based on the measurement data group, and exclude data determined to be the abnormal value from an alert target.

8. A pulse oximeter, comprising:
a green light sensor configured to irradiate a subject with green light and detect the green light transmitted through or reflected from the subject;
a red light sensor configured to irradiate the subject with red light and detect the red light transmitted through or reflected from the subject;
an infrared light sensor configured to irradiate the subject with infrared light and detect the infrared light transmitted through or reflected from the subject;
a control device configured to process detection signals from the green light sensor, the red light sensor and the infrared light sensor; and
a display unit configured to display information obtained by the control device, wherein
the control device is configured to
generate a measurement data group indicating an oxygen saturation of the subject based on the detection signals from the red light sensor and the infrared light sensor,
calculate a detection indicator of an abnormal value in the measurement data group based on the detection signals from the green light sensor, the red light sensor, and the infrared light sensor,
display the measurement data group and the detection indicator on the display unit on the same time axis,
display a predetermined threshold related to the detection indicator on the display unit, and
display a range where the detection indicator exceeds the threshold and a range where the detection indicator falls below the threshold in the measurement data group displayed on the display unit in different display formats.

9. The pulse oximeter according to claim 8, wherein
the control device is configured to display the threshold by a mark or a shape added to the display of the detection indicator.

10. The pulse oximeter according to claim 8, wherein
the control device is configured to display the range where the detection indicator exceeds the threshold and the range where the detection indicator falls below the threshold in different display formats using any one or a combination of saturation, brightness, and transparency.

11. The pulse oximeter according to claim 8, wherein
when the detection indicator exceeds the threshold, the control device is configured to display the measurement data group on the display unit and issue an alert regarding a posture of the subject.

12. A pulse oximeter, comprising:
a green light sensor configured to irradiate a subject with green light and detect the green light transmitted through or reflected from the subject;
a red light sensor configured to irradiate the subject with red light and detect the red light transmitted through or reflected from the subject;
an infrared light sensor configured to irradiate the subject with infrared light and detect the infrared light transmitted through or reflected from the subject;
a control device configured to process detection signals from the green light sensor, the red light sensor and the infrared light sensor; and
a display unit configured to display information obtained by the control device, wherein
the control device is configured to
generate a measurement data group indicating an oxygen saturation of the subject based on the detection signals from the red light sensor and the infrared light sensor,
calculate a detection indicator of an abnormal value in the measurement data group based on the detection signals from the green light sensor, the red light sensor, and the infrared light sensor, and
when the detection indicator exceeds a predetermined threshold, display the measurement data group on the display unit and issue an alert regarding a posture of the subject.
